(19) United States
(12) Patent Application Publication
(10) Pub. No.: US 2012/0051506 A1
(43) Pub. Date: Mar. 1, 2012

(54) RADIOLOGICAL IMAGE RADIOGRAPHING SYSTEM AND RADIOGRAPHING METHOD THEREOF

(75) Inventor: Kenji YOSHIKAWA, Kanagawa (JP)

(73) Assignee: Fujifilm Corporation, Tokyo (JP)

(21) Appl. No.: 13/221,765

(22) Filed: Aug. 30, 2011

(30) Foreign Application Priority Data
Aug. 31, 2010 (JP) P2010-193879
Jul. 21, 2011 (JP) P2011-159801

(51) Int. Cl. A61B 6/02

(52) U.S. Cl. 378/41

(57) ABSTRACT

S/N ratios of two radiological images with parallax are made equal. An angle between one of two radiographing directions and a direction perpendicular to a detection plane of a radiation detector is set to be larger than an angle between the other radiographing direction and the direction perpendicular to the detection plane of the radiation detector, and the amount of radiation is controlled such that the amount of radiation reaching the radiation detector from the one radiographing direction is equal to the amount of radiation reaching the radiation detector from the other radiographing direction.

### BACKGROUND OF THE INVENTION

1. Field of the Invention

The present invention relates to a radiological image radiographing system and its radiographing method for acquiring a stereoscopic image of a subject by radiating the subject from two different radiographing directions.

2. Description of the Related Art

In the related art, realizing stereoscopic viewing of a subject radiographed as a radiological image by displaying two radiological images with parallax in combination is known. Such two radiological images are generated based on signals detected by a radiation detector after radiating the same subject from two different radiographing directions.

In order to generate a high-quality radiological image, providing a grid which absorbs scattered light of radiation generated within the subject is known (JP2008-237631A).

In addition, an angle between two radiographing directions (hereinafter, referred to as a convergence angle) can be set as a desired angle and a combination of an angle (hereinafter, referred to as a radiographing angle), which is formed by each radiographing direction and a direction perpendicular to the detection plane of a radiation detector and which forms a predetermined convergence angle, may also be set in various ways.

### SUMMARY OF THE INVENTION

However, if the radiographing angle becomes large, vignetting due to the grid increases, and the amount of radiation reaching the radiation detector from a direction of a large radiographing angle decreases accordingly. For this reason, on a stereo image obtained by radiographing at different radiographing angles, the S/N ratio of a radiological image radiographed from a direction of a large radiographing angle is reduced compared with the S/N ratio of a radiological image radiographed from a direction of a small radiographing angle. Accordingly, since an observer views a stereo image formed by two radiological images with parallax which have different S/N ratios, the observer may feel more fatigued.

Therefore, in the radiological image radiographing system which radiates a subject from two different radiographing directions, the amount of radiated radiation needs to be controlled such that the amount of radiation reaching the radiation detector from each radiographing direction which is S (signal component), that is, the amount of arrival radiation becomes equal since N (noise component) of the S/N ratio is hardly influenced by the radiographing direction.

In view of the above problems, it is an object of the present invention to provide a radiological image radiographing system capable of radiographing two radiological images with parallax which have the same S/N ratio and its radiographing method.

In order to solve the above-described problem, according to an aspect of the present invention, there is provided a radiological image radiographing system including: a radiation source which radiates radiation to a subject from two different radiographing directions; a radiation detector which detects the radiated radiation; a grid which is disposed between the subject and the radiation detector to absorb scattered rays of the radiated radiation; radiation amount measuring portion for measuring the amount of radiation reaching the radiation detector; and radiation amount control portion for controlling the amount of radiation radiated from the radiation source. An angle between one of the two radiographing directions and a direction perpendicular to a detection plane of the radiation detector is larger than an angle between the other radiographing direction and the direction perpendicular to the detection plane of the radiation detector. The radiation amount control portion controls the amount of radiation such that the amount of radiation reaching the radiation detector from the one radiographing direction is equal to the amount of radiation reaching the radiation detector from the other radiographing direction.

Here, “the amounts of radiation are equal” means that the absolute value of a difference between two amounts of radiation falls within a range of ±15% of the average value of two amounts of radiation, and “S/N ratios are equal” means that the absolute value of a difference between S/N ratios of radiological images acquired in the two radiographing directions falls within a range of ±15% of the average value of the S/N ratios of radiological images acquired in the two radiographing directions.

Moreover, the radiological image radiographing system according to the present invention may further include exposure amount setting portion for setting the amount of exposure to the subject in advance. The radiation amount control portion may control the amount of radiation such that the amount of exposure of radiation from each of both the radiographing directions is equal to or smaller than the amount of exposure set in advance. Here, the “amount of exposure set in advance” means a sum of two amounts of radiation radiated to the subject.

Moreover, in the radiological image radiographing system according to the present invention, an angle between the two radiographing directions may be equal to or larger than 4° and equal to or smaller than 15°.

Moreover, in the radiological image radiographing system according to the present invention, an angle between the two radiographing directions may be 4°.

Moreover, in the radiological image radiographing system according to the present invention, the other radiographing direction may be parallel to the direction perpendicular to the detection plane.

Moreover, in the radiological image radiographing system according to the present invention, the exposure amount setting portion may calculate the amount of exposure, which is to be set in advance, based on the amount of radiation detected by the radiation detector after radiating a small amount of radiation from the direction perpendicular to the detection plane before radiating radiation to the subject from the different radiographing directions.

Here, the “small amount of radiation” means the amount of radiation radiated based on a small current product selected according to the subject thickness, for example, a tube current time product (mAs) of about 5 mAs when the radiological image radiographing system acquires a radiological image for diagnosis from the radiographing direction perpendicular to the detection plane through a single irradiation.

Moreover, the radiological image radiographing system according to the present invention may further include radiation quality control portion for controlling the quality of radiation radiated from the radiation source. The radiation quality control portion may control the quality of radiation radiated from the one radiographing direction to be harder than the quality of radiation radiated from the other radiographing direction.

Moreover, in the radiological image radiographing system according to the present invention, the radiation quality control portion may control the quality of radiation, which is radiated from the one radiographing direction, according to an angle between the one radiographing direction and the other radiographing direction.

Moreover, in the radiological image radiographing system according to the present invention, the radiation quality control portion may increase by 1 kVp a tube voltage of the radiation source when radiating radiation from the one radiographing direction whenever an angle between the one radiographing direction and the other radiographing direction increases by 1°.

In addition, according to another aspect of the present invention, a radiographing method of a radiological image radiographing system includes: radiating radiation to a subject from two different radiographing directions; detecting the radiated radiation using a radiation detector; absorbing scattered rays of the radiated radiation between the subject and the radiation detector; measuring the amount of radiation reaching the radiation detector; and controlling the amount of radiated radiation. An angle between one of the two radiographing directions and a direction perpendicular to a detection plane of the radiation detector is larger than an angle between the other radiographing direction and the direction perpendicular to the detection plane of the radiation detector. The amount of radiation is controlled such that the amount of radiation reaching the radiation detector from the one radiographing direction is equal to the amount of radiation reaching the radiation detector from the other radiographing direction.

In the radiological image radiographing system and its radiographing method according to the aspects of the present invention, the angle between one of the two radiographing directions and the direction perpendicular to the detection plane of the radiation detector is set to be larger than the angle between the other radiographing direction and the direction perpendicular to the detection plane of the radiation detector, and the amount of radiation is controlled such that the amount of radiation reaching the radiation detector from the one radiographing direction is equal to the amount of radiation reaching the radiation detector from the other radiographing direction. As a result, since the S/N ratios of radiological images acquired by both radiographing operations become equal, the fatigue of the observer can be reduced.

In addition, since the radiological image radiographing system according to the present invention includes the exposure amount setting portion for setting the amount of exposure to the subject in advance and the radiation amount control portion controls the amount of radiation such that the amount of exposure of radiation from each of both the radiographing directions is equal to or smaller than the amount of exposure set in advance, the S/N ratios of radiological images acquired by both radiographing operations can be made equal while suppressing the amount of exposure of the subject.

### BRIEF DESCRIPTION OF THE DRAWINGS

FIG. 1 is a view showing the schematic configuration of a radiological image radiographing system.

FIG. 2 is a front view of the radiological image radiographing system.

FIG. 3 is a view showing the internal configuration of a computer.

FIG. 4 is a view showing the internal configuration of a radiation source controller.

FIG. 5 is a flow chart showing an operation of the radiological image radiographing system.

FIG. 6 is a view showing the control of a radiation source controller.

### DESCRIPTION OF THE PREFERRED EMBODIMENTS

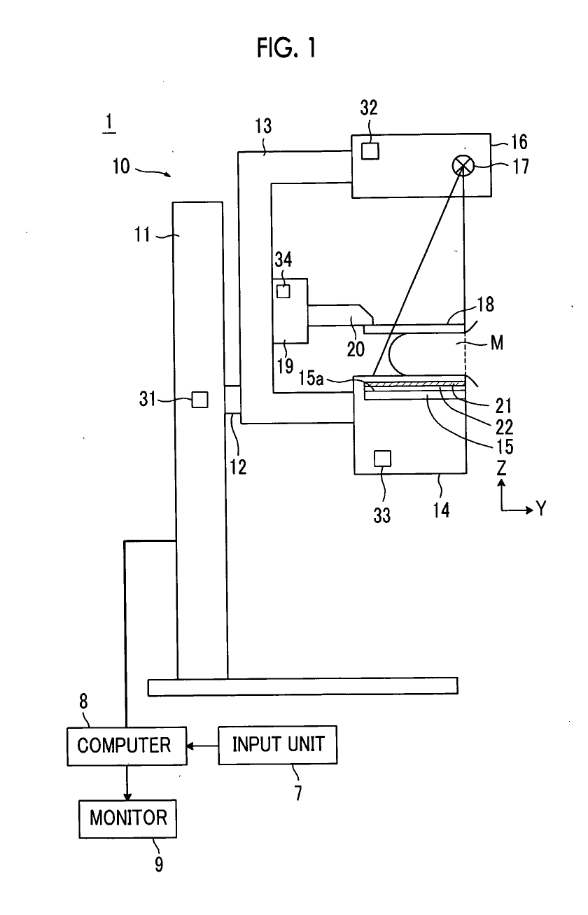

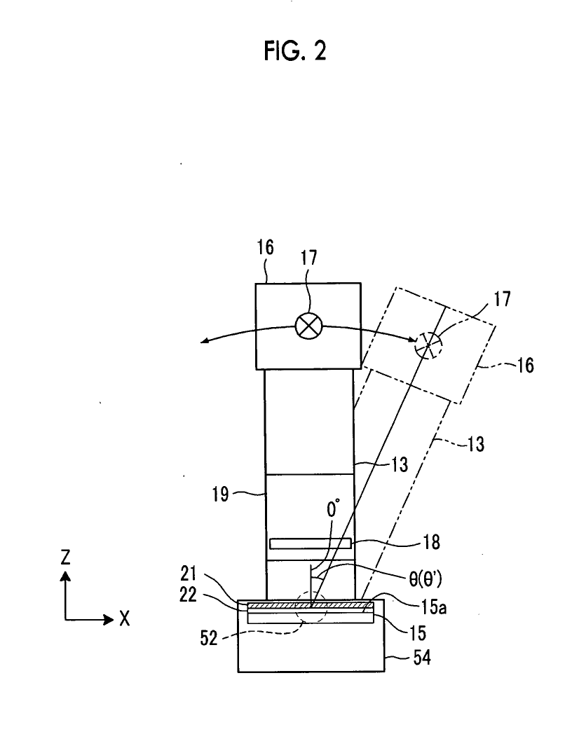

Hereinafter, embodiments of the present invention will be described with reference to the accompanying drawings. FIG. 1 is a view showing the schematic configuration of a radiological image radiographing system 1 according to an embodiment of the present invention, and FIG. 2 is a front view of the radiological image radiographing system 1.

As shown in FIG. 1, the radiological image radiographing system 1 includes a breast image radiographing apparatus 10, a computer 8 connected to the breast image radiographing apparatus 10, and a monitor 9 and an input unit 7 connected to the computer 8.

As shown in FIG. 1, the breast image radiographing apparatus 10 includes a pedestal 11, a rotary shaft 12 which can rotate and move up and down (in a Z direction) with respect to the pedestal 11, and an arm unit 13 connected to the pedestal 11 by the rotary shaft 12.

The arm unit 13 has a shape of a letter C. A radiation plane 14 is fixed to one end of the arm unit 13, and an irradiating unit 16 is fixed to the other end so as to face the radiation plane 14. Rotation and up-and-down movement of the arm unit 13 are controlled by an arm controller 31 provided in the pedestal 11.

A radiation detector 15, such as a flat panel detector, a grid 21 disposed on a detection plane 15a of the radiation detector 15, an AEC sensor 22 which is disposed between the grid 21 and the detection plane 15a to measure the amount of radiation reaching the detection plane 15a, and a detector controller 33 which controls reading of a charge signal from the radiation detector 15 are provided inside the radiation plane 14.

The grid 21 absorbs scattered light of radiation, and a member allowing radiation to be transmitted therethrough and a member not allowing radiation to be transmitted therethrough are alternately arrayed. The grid 21 is disposed so as to be inclined according to when radiation is radiated from a direction perpendicular to the detection plane 15a. Therefore, when the angle between a direction perpendicular to the detection plane 15a and a radiographing direction (hereinafter, referred to as a radiographing angle θ′) is large, vignetting by the grid 21 increases and the amount of arrival radiation decreases accordingly.

In addition, a circuit board on which a charge amplifier that converts a charge signal read from the radiation detector 15 into a voltage signal, a correlated double sampling circuit that samples a voltage signal output from the charge amplifier, an AD converter that converts a voltage signal into a digital signal, and the like are provided is placed inside the radiation plane 14.

In addition, the radiation plane 14 is configured to be able to rotate with respect to the arm unit 13. Accordingly, even when the arm unit 13 rotates with respect to the pedestal 11, the direction of the radiation plane 14 can be fixed with respect to the pedestal 11.

The radiation detector 15 can perform recording and reading of a radiological image repeatedly. A so-called direct conversion type radiological image detector which generates an electric charge by direct reception of radiation may be used, or a so-called indirect conversion type radiological image detector which converts radiation into visible light and then converts the visible light into a charge signal may be used.

Moreover, as a method of reading a radiological image signal, a so-called TFT reading method in which a radiological image signal is read by ON/OFF of a TFT (thin film transistor) switch or a so-called optical reading method in which a radiological image signal is read by irradiation of reading light is preferably used. However, other methods may be used without being limited to the above methods.

A radiation source 17 and a radiation source controller 32 are provided in the irradiating unit 16. The radiation source 17 radiates radiation with the center of the detection plane 15a of the radiation detector 15 as a focal point. The radiation source controller 32 controls an irradiation timing of radiation from the radiation source 17 and the radiation generating conditions (tube current (mA), irradiation time (ms), tube current time product (mAs), tube voltage (kV), and the like) in the radiation source 17. Details of the configuration of the radiation source controller 32 will be described later.

In addition, a compression plate 18 provided above the radiation plane 14 to compress a breast, a supporting unit 20 which supports the compression plate 18, and a moving mechanism 19 which moves the supporting unit 20 up and down (in the Z direction) are provided in the middle of the arm unit 13. The position and the pressure of the compression plate 18 are controlled by the compression plate controller 34.

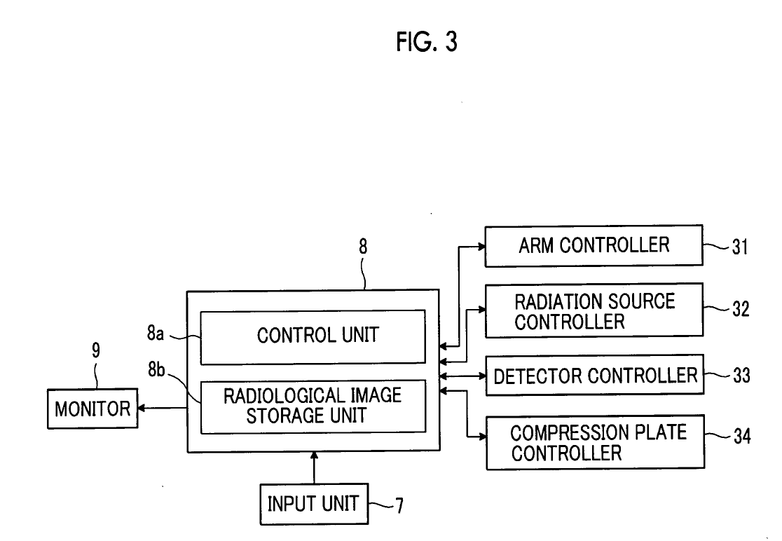

The computer 8 includes a central processing unit (CPU) and a storage device, such as a semiconductor memory, a hard disk, or an SSD. By such hardware, a controller 8a and a radiological image storage unit 8b shown in FIG. 3 are formed.

The controller 8a outputs predetermined control signals to various kinds of controllers 31 to 34 to control the entire system. A specific control method will be described in detail later.

The radiological image storage unit 8b stores a radiological image signal in each radiographing direction, which is acquired by the radiation detector 15, in advance.

The input unit 7 includes a keyboard or a pointing device, such as a mouse, and receives from a radiographer an input of radiographing conditions, an input of an operation instruction, and the like.

The monitor 9 is configured to display a stereo image by displaying a radiological image in each radiographing direction as a two-dimensional image using two radiological image signals output from the computer 8.

As an example of the configuration for displaying a stereo image, a configuration may be adopted in which radiological images based on two radiological image signals are displayed using two corresponding screens and a half mirror, polarizing glasses, and the like are used to make one of the radiological images incident on the right eye of an observer and the other radiological image incident on the left eye of the observer.

Alternatively, for example, a configuration may be adopted in which a stereo image is generated by displaying two radiological images so as to overlap each other by shifting them from each other by the predetermined amount of parallax and making the overlapped image be observed through the polarizing glasses, or a configuration may be adopted in which a stereo image is generated by displaying two radiological images on a 3D display through which stereoscopic viewing is possible like a parallax barrier method and a lenticular method. In addition, a device which displays a stereo image and a device which displays a two-dimensional image may be separately configured or may be configured as the same device when a stereo image and a two-dimensional image can be displayed on the same screen.

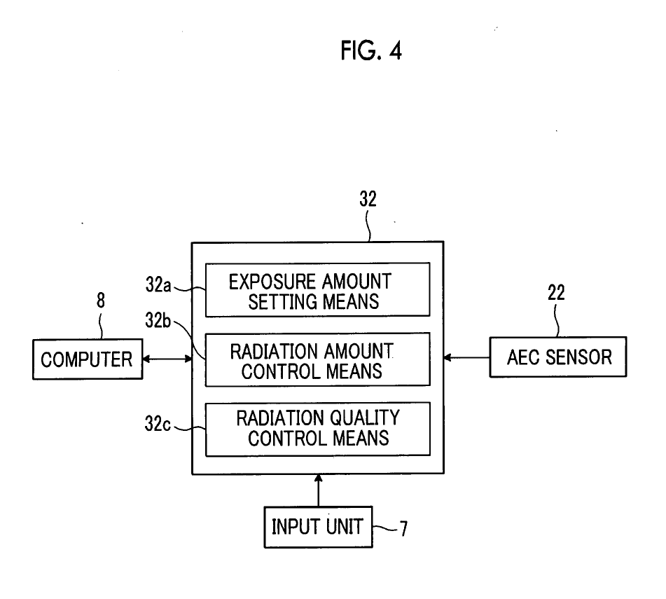

As described above, the radiation source controller 32 controls an irradiation timing of radiation from the radiation source 17 and the radiation generating conditions (tube current (mA), irradiation time (ms), tube current time product (mAs), tube voltage (kV), and the like) in the radiation source 17. As shown in FIG. 4, the radiation source controller 32 includes exposure amount setting portion 32a, radiation amount control portion 32b, and radiation quality control portion 32c.

The exposure amount setting portion 32a sets the amount of exposure of a subject in advance by performing preliminary radiographing with a small amount of radiation before radiographing. Here, the small amount of radiation used for preliminary radiographing is radiation the amount of which is about 5% of the amount of radiation when performing radiographing for normal diagnosis through a single irradiation from the direction perpendicular to the detection plane 15a.

The radiation amount control portion 32b controls the amount of radiation, which is radiated from the radiation source 17, by controlling a tube current and irradiation time of the radiation source 17 based on the information from the AEC sensor 22.

The radiation quality control portion 32c controls the quality of radiation, which is radiated from the radiation source 17, by controlling a tube voltage of the radiation source 17.

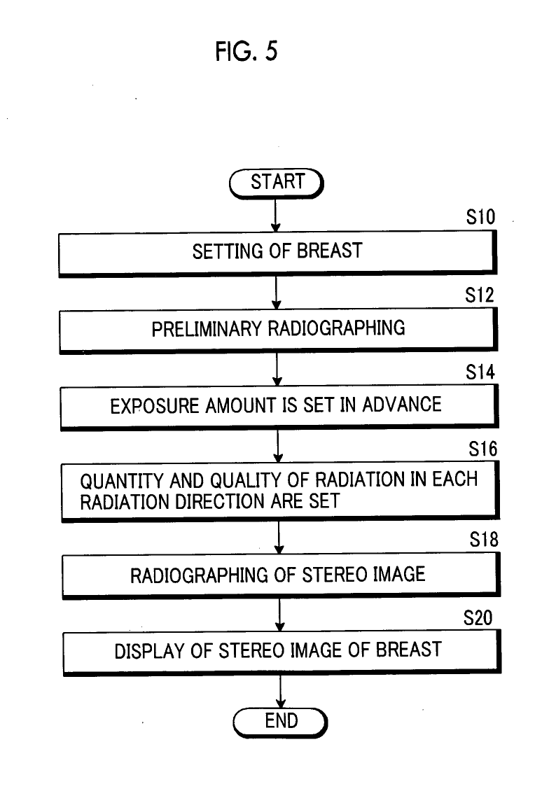

An operation of the radiological image radiographing system 1 will be described with reference to FIG. 5. First, a breast M is placed on the radiation plane 14 and is compressed with predetermined pressure by the compression plate 18 (S10). Then, in order to set the amount of exposure LE to the breast M in advance, an instruction to start preliminary radiographing is input through the input unit 7 by a radiographer.

Then, if there is an instruction to start preliminary radiographing through the input unit 7, preliminary radiographing is performed (S12). Specifically, first, the controller 8a reads the radiographing angle θ′ for preliminary radiographing and outputs the information regarding the read radiographing angle to the arm controller 31. In addition, in the present embodiment, θ′=0° is assumed to be stored in advance as the information regarding the radiographing angle θ′.

In addition, the arm controller 31 receives the information regarding the radiographing angle θ′ output from the controller 8a and outputs a control signal to make the arm unit 13 positioned in a direction perpendicular to the detection plane 15a as shown in FIG. 3.

Then, in a state where the arm unit 13 is positioned perpendicular to the detection plane 15a according to the control signal output from the arm controller 31, the controller 8a outputs a control signal to the radiation source controller 32 and the detector controller 33 in order to perform irradiation and reading of a radiological image.

According to this control signal, radiation is radiated from the radiation source 17, radiation used when radiographing the breast M from a direction of the radiographing angle θ′ of 0° is detected by the radiation detector 15, and a radiological image signal is read from the radiation detector 15 by the detector controller 33.

Then, the controller 8a outputs to the radiation source controller 32 a control signal to set the amount of exposure LE of the breast M in radiographing. The exposure amount setting portion 32a sets the amount of exposure LE in radiographing in advance by calculating the amount of exposure to the breast M based on the read radiological image signal (S14).

Then, various radiographing conditions including a combination of an angle formed by two different radiographing directions (hereinafter, referred to as a convergence angle θ) and the radiographing angle θ′, which forms the convergence angle θ, are input to the input unit 7, and then an instruction to start radiographing is input to the input unit 7.

Then, the controller 8a outputs to the radiation source controller 32 a control signal to set the amount and the quality of radiation radiated in each radiographing direction. The radiation amount control portion 32b and the radiation quality control portion 32c set the amount and the quality of radiation radiated in each radiographing direction based on the amount of exposure LE, the convergence angle θ, and the radiographing angle θ′ (S16). In addition, control of the amount and the quality of radiation from the radiation source 17 performed by the radiation source controller 32 will be described later.

Then, if there is an instruction to start radiographing through the input unit 7, radiographing of a stereo image of the breast M is performed (S18). Specifically, first, the controller 8a outputs to the arm controller 31 the information regarding the convergence angle θ and the radiographing angle θ′ which forms the convergence angle θ. In addition, although θ=4° is set as the information regarding the convergence angle θ and a combination of θ′=0° and θ′=4° is set as the combination of the radiographing angle θ′ which forms the convergence angle θ in the present embodiment, a radiographer can set an arbitrary convergence angle θ through the input unit 7 without being limited to this. The convergence angle θ is preferably set to 4° or more and 15° or less. In addition, the combination of the radiographing angle θ′ is not particularly limited as long as one radiographing angle θ′ is larger than the other radiographing angle θ′. In addition, as the combination of the radiographing angle θ′, it is preferable to set the other radiographing angle θ′ to 0°.

The arm controller 31 receives the information regarding the convergence angle θ output from the controller 8a and outputs a control signal to make the arm unit 13 positioned in a direction perpendicular to the radiation plane 14 based on the information regarding the convergence angle θ. That is, in the present embodiment, the arm controller 31 outputs a control signal to set the radiographing angle θ′ to 0° so that the arm unit 13 is positioned in a direction perpendicular to the detection plane 15a.

According to the control signal output from the arm controller 31, the arm unit 13 is positioned perpendicular to the detection plane 15a. Then, the controller 8a outputs to the radiation source controller 32 and the detector controller 33 a control signal to perform irradiation and reading of a radiological image signal. According to this control signal, radiation is radiated from the radiation source 17, a radiological image obtained by radiographing the breast M from a direction of the radiographing angle θ′ of 0° is detected by the radiation detector 15, a radiological image signal is read by the detector controller 33, and predetermined signal processing is performed on the radiological image signal. Then, the radiological image signal is stored in the radiological image storage unit 8b of the computer 8.

The arm controller 31 outputs a control signal to make the arm unit 13 rotate by 4° in a direction perpendicular to the detection plane 15a as shown in FIG. 2. That is, in the present embodiment, the arm controller 31 outputs a control signal to make the arm unit 13 be inclined by the radiographing angle θ′ of 4° with respect to the direction perpendicular to the detection plane 15a.

According to the control signal output from the arm controller 31, the arm unit 13 rotates by 4°. Then, the controller 8a outputs to the radiation source controller 32 and the detector controller 33 a control signal to perform irradiation and reading of a radiological image. According to this control signal, radiation is radiated from the radiation source 17, a radiological image obtained by radiographing the breast M from a direction of the radiographing angle θ′ of 4° is detected by the radiation detector 15, a radiological image signal is read by the detector controller 33, and predetermined signal processing is performed. Then, the radiological image signal is stored in the radiological image storage unit 8b of the computer 8.

The two radiological image signals stored in the radiological image storage unit 8b of the computer 8 are read from the radiological image storage unit 8b, and then predetermined signal processing is performed on the two radiological image signals and the result is output to the monitor 9. Accordingly, a stereo image of the breast M is displayed on the monitor 9 (S20).

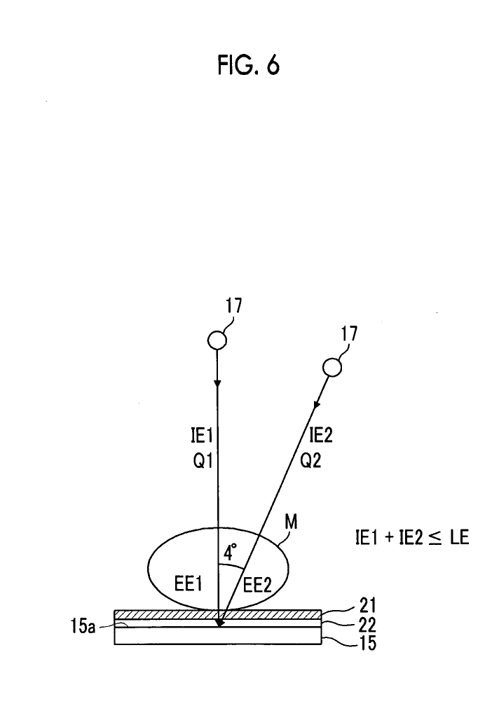

Then, control of the radiation source controller 32 will be described. FIG. 6 is a view showing the control of the radiation source controller 32. The radiation source controller 32 at the time of radiographing controls the amount and the quality of radiation, which is radiated from the radiation source 17, in each radiographing direction, based on the amount of exposure LE, the convergence angle θ, and the radiographing angle θ′ which forms the convergence angle θ. In addition, in the first embodiment, the quality of radiation is assumed to be constant irrespective of the radiographing direction.

Moreover, in each of the following embodiments, the amount of exposure LE will be described as a limit value of the sum of the amounts of radiation radiated from two radiographing directions. Specifically, in the present embodiment, the amount of exposure LE set in advance is 220 mR.

The radiation amount control portion 32b controls the amounts of radiation IE1 and IE2, which is radiated from the radiation source 17, in each radiographing direction based on the amount of exposure LE, the two radiograph angles θ′ which form the convergence angle θ, and the information from the radiation quality control portion 32c.

A table showing the ratio of the amount of arrival radiation to the amount of radiated radiation based on the amount of absorption in the breast M corresponding to the radiographing angle θ′, the amount of vignetting at the grid 21, and the like is stored in advance in the radiation amount control portion 32b. In the present embodiment, the radiation amount control portion 32b reads that the amount of arrival radiation EE1 when the radiographing angle θ′ is 0° is about 70% of the amount of radiated radiation IE1 and the amount of arrival radiation EE2 when the radiographing angle θ′ is 4° is about 40% of the amount of radiated radiation IE2 using this table.

Based on the ratio of the amount of arrival radiation to the amount of radiated radiation and the amount of arrival radiation that the radiographer wants, the radiation amount control portion 32h controls the amounts of radiated radiation IE1 and IE2 in each radiographing direction such that the amounts of arrival radiation EE1 and EE2 become equal. Here, “the amounts of arrival radiation EE1 and EE2 are equal” refers to performing control such that the absolute value of a difference between the amounts of arrival radiation EE1 and EE2 falls within a range of ±15% of the average value of the amounts of arrival radiation EE1 and EE2. In addition, the amount of arrival radiation that a radiographer wants is input as a radiographing condition through the input unit 7. In the present embodiment, the amount of arrival radiation of 55 mR is assumed to be input.

In this case, the radiation amount control portion 32b sets the amount of arrival radiation that a radiographer wants such that the sum of the amounts of radiated radiation IE1 and IE2 becomes equal to or smaller than the set amount of exposure LE.

In the present embodiment, the amount of arrival radiation EE1 becomes 55 mR by controlling the amount of radiated radiation IE1 when the radiographing angle θ′ is 0° so as to become 786 mR, and the amount of arrival radiation EE2 also becomes 55 mR by controlling the amount of radiated radiation IE2 when the radiographing angle θ′ is 4° so as to become 137.5 mR.

The radiation amount control portion 32b radiates radiation, the amount of which is IE1 and IE2, from the radiation source 17 in each radiographing direction and also controls the radiation source 17 such that the predetermined amounts of arrival radiation EE1 and EE2 are obtained based on the information from the AEC sensor 22. Specifically, the radiation amount control portion 32b controls a tube current (mA) and irradiation time (mS) of the radiation source 17.

Accordingly, since the amounts of arrival radiation EE1 and EE2 become equal when the radiographing angle θ′ is 0° and when the radiographing angle θ′ is 4°, the S/N ratio of a radiological image signal from the radiation detector 15 in each radiographing direction becomes equal. In addition, the sum of the amounts of arrival radiation EE1 and EE2 is also 216.1 mR, which is equal to or smaller than the set amount of exposure LE. Therefore, the amount of exposure to the breast M is also suppressed.

Next, a second embodiment of the control of the radiation source controller 32 will be described. The second embodiment is different from the first embodiment in that the quality of radiation radiated in radiographing when the radiographing angle θ′ is large is set to be harder than the quality of radiation radiated in radiographing when the radiographing angle θ′ is small.

A table showing the S/N ratio of a radiological image signal corresponding to the radiographing angle θ′, the amount of radiated radiation, and the quality of radiated radiation is stored in the radiation quality control portion 32c. Specifically, a tube current product (mAs) is stored for the amount of radiated radiation, and a tube voltage (kV) is stored for the quality of radiated radiation. In addition, a table showing a combination of a tube current product (mAs) and a tube voltage (kV) satisfying a predetermined S/N ratio is also stored in the radiation quality control portion 32c.

Generally, in the combination of the amount of radiated radiation and the quality of radiated radiation satisfying a predetermined S/N ratio, the amount of radiated radiation can be reduced in proportion to making the quality of radiated radiation hard (increasing the tube voltage). In the second embodiment, since the quality of radiated radiation Q2′ when the radiographing angle θ′ is 4° is made to be harder than the quality of radiated radiation Q2 in the first embodiment, the amount of radiated radiation IE2′ is reduced compared with the amount of radiated radiation IE2 in the first embodiment. As a result, the S/N ratio of a radiological image signal in each radiographing direction can be made equal to that in the first embodiment. Moreover, in the second embodiment, the S/N ratio in the combination of the quality of radiated radiation Q2′ and the amount of radiated radiation IE2′ when the radiographing angle θ′ is 4° can be improved more than that when the radiographing angle θ′ is 4° in the first embodiment.

In addition, the amount of radiated radiation IE2′ by which the amount of arrival radiation IE2′ becomes about 55 mR is calculated based on the quality of radiated radiation Q2′ hardened when the radiographing angle θ′ is 4°. In addition, the amount of radiated radiation IE1′ and the quality of radiated radiation Q1′ when the radiographing angle θ′ is 0° are the same as the amount of radiated radiation IE1 and the quality of radiated radiation Q1 in the first embodiment.

Specifically, the quality of radiated radiation Q2′ is made harder than the quality of radiated radiation Q2 by setting a tube voltage, which is related to the quality of radiated radiation Q2′ in the second embodiment, to be higher by about 10% than a tube voltage, which is related to the quality of radiated radiation Q2 in the first embodiment. Accordingly, the amount of absorption in the breast M and the ratio of the amount of arrival radiation to the amount of radiated radiation due to vignetting at the grid 21 when the radiographing angle θ′ is 4° also increase from 40% to 50%. It is assumed that the radiation amount control portion 32b stores in advance a table showing the ratio of the amount of arrival radiation to the amount of radiated radiation according to the quality of radiated radiation. The radiation amount control portion 32b performs control to reduce the amount of radiated radiation IE2′ when the radiographing angle θ′ is 4° to about 110 mR, so that the amount of arrival radiation EE2′ becomes about 55 mR. In addition to increasing the tube voltage, the quality of radiated radiation may also be hardened by changing the combination of a target filter.

When the radiographing angle θ′ is 4°, IE2′ becomes 110 mR and the amount of arrival radiation EE2′ becomes 55 mR by making the quality of radiated radiation Q2′ hard. Accordingly, the S/N ratio of a radiological image signal in each radiographing direction can be made equal to that in the first embodiment, and the sum of IE1′ and IE2′ becomes 188.6 mR. Thus, since the sum of IE1′ and IE2′ is equal to or smaller than the amount of exposure LE and is also smaller than that in the first embodiment, the amount of exposure to the breast M is suppressed.

In addition, in the second embodiment, the radiation quality control portion 32c may control the quality of radiated radiation Q2′ according to the value of the convergence angle θ. Specifically, the tube voltage of the radiation source 17 may be set to increase by 1 kVp whenever the convergence angle θ increases by 1°. Moreover, in the second embodiment, the contrast of a radiological image when the radiographing angle θ′ is 4° becomes lower than the contrast of a radiological image when the radiographing angle θ′ is 0° by making the quality of radiated radiation Q2′ harder than the quality of radiated radiation Q1′. Accordingly, image processing may be performed such that the contrast of both the radiological images becomes equal when displaying the image on the monitor 9.

As described above, according to the radiological image radiographing system 1, the S/N ratio of two radiological images with parallax can be made equal by controlling the amount of radiation in each radiographing direction such that the amounts of arrival radiation EE1 and EE2 become equal to each other. As a result, the fatigue of an observer can be reduced.

In addition, according to the radiological image radiographing system 1, the S/N ratio of two radiological images with parallax can be made equal while suppressing the amount of exposure by controlling the amount of radiation in each radiographing direction so as to be equal to or smaller than the amount of exposure LE set in advance.

In addition, although the breast image radiographing system is used as the radiological image radiographing system according to the embodiment of the present invention, a subject is not limited to the breast. For example, a radiological image radiographing system which radiographs a chest, a head, and the like may also be used.

### First Example

In the radiological image radiographing apparatus 1, verification related to increasing the tube voltage (kV) was performed as follows. Table 1 shows a result of this verification. Table 1 shows the radiographing angle θ′, the tube voltage (kV), the tube current product (mAs), and the S/N ratio in each radiographing condition.

In this example, radiographing conditions (A) in which both tube voltages (kV) are 28 kV, the radiographing angle θ′ is 0° and 4°, and both tube current products indicating the amount of exposure (mAs) are 36 mAs are adopted as the original radiographing conditions in the radiological image radiographing apparatus 1. In the radiographing conditions (A), the S/N ratio was set to 143.6 and 123.2. In addition, the amount of exposure LE was set to 75 mAs in advance.

In addition, by changing the radiographing conditions to radiographing conditions (B) in which only the tube current (kV) when the radiographing angle θ′ was 4° was increased to 32 kV in order to increase the S/N ratio when the radiographing angle θ′ was 4° although the radiographing conditions (A) when the radiographing angle θ′ was 0° were maintained as they were, the S/N ratio when the radiographing angle θ′ was 4° was increased to 194.2. In this case, since both the tube current products (mAs) are 36 mAs, there is no difference in the amount of exposure between the radiographing conditions (A) and the radiographing conditions (B).

However, since the average value of the S/N ratios is 168.9 and the difference between the S/N ratios is 50.6, a difference from the average value is 30%. Accordingly, a difference between both the S/N ratios increases significantly. For this reason, by changing the radiographing conditions to radiographing conditions (C) in which the radiographing conditions when the radiographing angle θ′ was 0° were the same as the radiographing conditions (A) but the tube current (kV) when the radiographing angle θ′ was 4° was set to 32 kV in the same manner as in the radiographing conditions (B) and the tube current product (mAs) was reduced to 28 mAs, the S/N ratio when the radiographing angle θ′ was 4° became 155.3. Accordingly, since the average value of the S/N ratios became 149.45 and the difference between the S/N ratios became 11.7, a difference from the average value became about 7.8%. As a result, it was confirmed that both the S/N ratios could be made equal and the amount of exposure was more suppressed in the radiographing conditions (C) than in the radiographing conditions (A).

Then, by changing the radiographing conditions to radiographing conditions (D) in which only the tube current product (mAs) when the radiographing angle θ′ was 0° was increased to 45 mAs in a range not exceeding the amount of exposure LE in proportion to the amount of reduction in the tube current product (mAs) when the radiographing angle θ′ was 4° and the radiographing conditions (C) when the radiographing angle θ′ was 4° were maintained as they were, the S/N ratio when the radiographing angle θ′ was 0° could be increased to 167.0. Accordingly, the average value of the S/N ratios was increased to 161.15, and the difference between the S/N ratios became 11.7. As a result, since a difference from the average value became about 7.3%, it was confirmed that both the S/N ratios could be made more equal.

TABLE 1

| Radiographing | | | | |
|---|---|---|---|---|
| conditions | Angle | mAs | kV | S/N ratio |
| A | 0 | 36 | 28 | 143.6 |
| | 4 | 36 | 28 | 123.2 |
| B | 0 | 36 | 28 | 143.6 |
| | 4 | 36 | 32 | 194.2 |
| C | 0 | 36 | 28 | 143.6 |
| | 4 | 28 | 32 | 155.3 |
| D | 0 | 45 | 28 | 167 |
| | 4 | 28 | 32 | 155.3 |

What is claimed is:

1. A radiological image radiographing system comprising:
a radiation source which radiates radiation to a subject from two different radiographing directions;
a radiation detector which detects the radiated radiation;
a grid which is disposed between the subject and the radiation detector to absorb scattered rays of the radiated radiation;
radiation amount measuring portion for measuring the amount of radiation reaching the radiation detector; and
radiation amount control portion for controlling the amount of radiation radiated from the radiation source,
wherein an angle between one of the two radiographing directions and a direction perpendicular to a detection plane of the radiation detector is larger than an angle between the other radiographing direction and the direction perpendicular to the detection plane of the radiation detector, and
the radiation amount control portion controls the amount of radiation such that the amount of radiation reaching the radiation detector from the one radiographing direction is equal to the amount of radiation reaching the radiation detector from the other radiographing direction.

2. The radiological image radiographing system according to claim 1, further comprising:
exposure amount setting portion for setting the amount of exposure to the subject in advance,
wherein the radiation amount control portion controls the amount of radiation such that the amount of exposure of radiation from each of both the radiographing directions is equal to or smaller than the amount of exposure set in advance.

3. The radiological image radiographing system according to claim 1,
wherein an angle between the two radiographing directions is equal to or larger than 4° and equal to or smaller than 15°.

4. The radiological image radiographing system according to claim 2,
wherein an angle between the two radiographing directions is equal to or larger than 4° and equal to or smaller than 15°.

5. The radiological image radiographing system according to claim 1,
wherein an angle between the two radiographing directions is 4°.

6. The radiological image radiographing system according to claim 2,
wherein an angle between the two radiographing directions is 4°.

7. The radiological image radiographing system according to claim 1,
wherein the other radiographing direction is parallel to the direction perpendicular to the detection plane.

8. The radiological image radiographing system according to claim 2,
wherein the other radiographing direction is parallel to the direction perpendicular to the detection plane.

9. The radiological image radiographing system according to claim 3,
rein the other radiographing direction is parallel to the direction perpendicular to the detection plane.

10. The radiological image radiographing system according to claim 4,
wherein the other radiographing direction is parallel to the direction perpendicular to the detection plane.

11. The radiological image radiographing system according to claim 2,
wherein the exposure amount setting portion calculates the amount of exposure, which is to be set in advance, based on the amount of radiation detected by the radiation detector after radiating a small amount of radiation from the direction perpendicular to the detection plane before radiating radiation to the subject from the different radiographing directions.

12. The radiological image radiographing system according to claim 1, further comprising:
radiation quality control portion for controlling the quality of radiation radiated from the radiation source,
wherein the radiation quality control portion controls the quality of radiation radiated from the one radiographing direction to be harder than the quality of radiation radiated from the other radiographing direction.

13. The radiological image radiographing system according to claim 2, further comprising:
radiation quality control portion for controlling the quality of radiation radiated from the radiation source,
wherein the radiation quality control portion controls the quality of radiation radiated from the one radiographing direction to be harder than the quality of radiation radiated from the other radiographing direction.

14. The radiological image radiographing system according to claim 3, further comprising:
radiation quality control portion for controlling the quality of radiation radiated from the radiation source,
wherein the radiation quality control portion controls the quality of radiation radiated from the one radiographing direction to be harder than the quality of radiation radiated from the other radiographing direction.

15. The radiological image radiographing system according to claim 4, further comprising:
radiation quality control portion for controlling the quality of radiation radiated from the radiation source,
wherein the radiation quality control portion controls the quality of radiation radiated from the one radiographing direction to be harder than the quality of radiation radiated from the other radiographing direction.

16. The radiological image radiographing system according to claim 12,
wherein the radiation quality control portion controls the quality of radiation, which is radiated from the one radiographing direction, according to an angle between the one radiographing direction and the other radiographing direction.

17. The radiological image radiographing system according to claim 13,
wherein the radiation quality control portion controls the quality of radiation, which is radiated from the one radiographing direction, according to an angle between the one radiographing direction and the other radiographing direction.

18. The radiological image radiographing system according to claim 12,
wherein the radiation quality control portion increases by 1 kVp a tube voltage of the radiation source when radiating radiation from the one radiographing direction whenever an angle between the one radiographing direction and the other radiographing direction increases by 1°.

19. The radiological image radiographing system according to claim 13,
wherein the radiation quality control portion increases by 1 kVp a tube voltage of the radiation source when radiating radiation from the one radiographing direction whenever an angle between the one radiographing direction and the other radiographing direction increases by 1°.

20. A radiographing method of a radiological image radiographing system comprising:
radiating radiation to a subject from two different radiographing directions;
detecting the radiated radiation using a radiation detector;
absorbing scattered rays of the radiated radiation between the subject and the radiation detector;
measuring the amount of radiation reaching the radiation detector; and
controlling the amount of radiated radiation,
wherein an angle between one of the two radiographing directions and a direction perpendicular to a detection plane of the radiation detector is larger than an angle between the other radiographing direction and the direction perpendicular to the detection plane of the radiation detector, and
the amount of radiation is controlled such that the amount of radiation reaching the radiation detector from the one radiographing direction is equal to the amount of radiation reaching the radiation detector from the other radiographing direction.
